(19) United States
(12) Patent Application Publication
(10) Pub. No.: US 2007/0232628 A1
(43) Pub. Date: Oct. 4, 2007

(54) Calcilytic Compounds

(76) Inventors: Juan I. Luengo, Collegeville, PA (US); Robert W. Marquis, Collegeville, PA (US); Ren Xie, Collegeville, PA (US); Dennis S. Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/568,709

(22) PCT Filed: May 3, 2005
(86) PCT No.: PCT/US05/15224

§ 371 Date: Nov. 6, 2006

Related U.S. Application Data
(60) Provisional application No. 60/568,585, filed on May 6, 2004.

(51) Int. Cl. A61K 31/505; A61P 19/00; A61P 19/02; A61P 19/08; A61P 43/00; C07D 237/26; C07D 239/00; C07D 239/02; C07D 471/04; C07D 487/00

(52) U.S. Cl. 514/266.3; 514/257; 544/235; 544/249; 544/283; 544/317

(57) ABSTRACT

Novel calcilytic compounds and methods of using them are provided.

### FIELD OF INVENTION

The present invention relates to novel calcilytic compounds, pharmaceutical compositions containing these compounds and their use as calcium receptor antagonists.

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evoked by extracellular Ca²⁺. Calcilytics are useful as lead molecules in the discovery, development, design, modification and/or construction of useful calcium modulators, which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

### SUMMARY OF THE INVENTION

The present invention comprises novel calcium receptor antagonists represented by Formula (I) hereinbelow and their use as calcium receptor antagonists in the treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

The present invention further provides a method for antagonizing calcium receptors in an animal, including humans, which comprises administering to an animal in need thereof an effective amount of a compound of Formula (I), indicated hereinbelow.

The present invention further provides a method for increasing serum parathyroid levels in an animal, including humans, which comprises administering to an animal in need thereof an effective amount of a compound of Formula (I), indicated herein below.

### DETAILED DESCRIPTION OF THE INVENTION

The compounds of the present invention are selected from Formula (I) herein below: 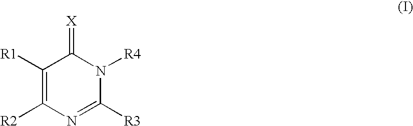
wherein:
R1 and R2 may each be independently selected from the group consisting of H, halogen, CN, alkyl, alkyl-aryl, aryl, substituted aryl, hetero aryl and substituted heteroaryl
   or R1 and R2 may be bonded together to form a carbocylic ring, heterocylic ring, aryl or heteroaryl ring
   R3 is an aryl group or heteroaryl group which may have 1-5 substituents each selected from the group consisting of H, halogen, CN, CF₃, OCF₃, alkyl, alkoxy, OC(O)alkyl or OH
   R4 is an aryl group which may have 1 to 3 substituents consisting of H, halogen, CN, CF₃, alkyl, substituted alkyl and alkoxy; and
   X is oxygen or sulphur.

As used herein, “alkyl” refers to an optionally substituted hydrocarbon group joined by single carbon-carbon bonds and having 1-20 carbon atoms joined together. The alkyl hydrocarbon group may be linear, branched or cyclic, saturated or unsaturated. Preferably, substituents on optionally substituted alkyl are selected from the group consisting of aryl, CO₂R, CO₂NHR, OH, OR, CO, NH₂, halo, CF₃, OCF₃ and NO₂, wherein R represents H, C₁₋₄ alkyl, C₃₋₆ cycloalkyl, C₂₋₅ alkenyl, C₂₋₅ alkynyl, heterocycloalkyl, or aryl. Additional substituents are selected from F, Cl, Br, I, N, S and O. Preferably, no more than three substituents are present. More preferably, the alkyl has 1-12 carbon atoms and is unsubstituted. Preferably, the alkyl group is linear.

As used herein “cycloalkyl” refers to optionally substituted 3-7 membered carbocyclic rings wherein any substituents are selected from the group consisting of, F, Cl, Br, I, N(R₄)₂, SR₄ and OR₄, unless otherwise indicated.

As used herein, “aryl” refers to an optionally substituted aromatic group with at least one ring having a conjugated pi-electron system, containing up to two conjugated or fused ring systems. Aryl includes carbocyclic aryl, and biaryl groups, all of which may be optionally substituted. Preferred aryl include phenyl and naphthyl. More preferred aryl include phenyl. Preferred substituents are selected from the group consisting of halogen, C₁₋₄ alkyl, OCF₃, CF₃, OMe, CN, OSO₂R and NO₂, wherein R represents C₁₋₄ alkyl or C₃₋₆ cycloalkyl.

As used herein, “heteroaryl” refers to an aryl ring containing 1, 2 or 3 heteroatoms such as N, S, or O.

As used herein, “alkenyl” refers to an optionally substituted hydrocarbon group containing at least one carbon-carbon double bond and containing up to 5 carbon atoms joined together. The alkenyl hydrocarbon chain may be straight, branched or cyclic. Any substituents are selected from the group consisting of halogen, C₁₋₄ alkyl, OCF₃, CF₃, OMe, CN, OSO₂R and NO₂, wherein R represents C₁₋₄ alkyl or C₃₋₆ cycloalkyl.

As used herein, “alkynyl” refers to an optionally substituted hydrocarbon group containing at least one carbon-carbon triple bond between the carbon atoms and containing up to 5 carbon atoms joined together. The alkynyl hydrocarbon group may be straight-chained, branched or cyclic. Any substituents are selected from the group consisting of halogen, C₁₋₄ alkyl, OCF₃, CF₃, OMe, CN, OSO₂R and NO₂, wherein R represents C₁₋₄ alkyl or C₃₋₆ cycloalkyl.

The compounds of the present invention may contain one or more asymmetric carbon atoms and may exist in racemic and optically active forms. All of these compounds and diastereomers are contemplated to be within the scope of the present invention.

Preferred compounds of the present inventions include:
2-(2-hydroxyphenyl)-3-[4-(1-methylethyl)phenyl]-5,6,7,8-tetrahydro-4(3H)-quinazolinone;
5-ethyl-2-(2-hydroxyphenyl)-6-methyl-3-[4-(1-methylethyl)phenyl]-4(3H)-pyrimidinone;
5-ethyl-2-(3-fluoro-2-hydroxyphenyl)-6-methyl-3-[4-(1-methylethyl)phenyl]-4(3)-pyrimidinone;
2-(2-hydroxyphenyl)-6-methyl-3-[4-(1-methylethyl)phenyl]-5-(2-methylpropyl)-4(3)-pyrimidinone;
2-(3-fluoro-2-hydroxyphenyl)-6-methyl-3-[4-(1-methylethyl)phenyl]-5-(2-methylpropyl)-4(3H)-pyrimidinone;
2-(2-hydroxyphenyl)-6-methyl-3-[4-(1-methylethyl)phenyl]-4(3H)-pyrimidinone;
2-(2-hydroxyphenyl)-5,6-dimethyl-3-[4-(1-methylethyl)phenyl]-4(3H)-pyrimidinone;
2-(2-hydroxyphenyl)-6-methyl-3-[4-(1-methylethyl)phenyl]-5-propyl-4(3H)-pyrimidinone;
5-butyl-2-(2-hydroxyphenyl)-6-methyl-3-[4-(1-methylethyl)phenyl]-4(3H)-pyrimidinone;
2-(2-hydroxyphenyl)-6-methyl-3-[4-(1-methylethyl)phenyl]-5-phenyl-4(3H)-pyrimidinone;
5-(1-benzothien-2-yl)-2-(2-hydroxyphenyl)-6-methyl-3-[4-(1-methylethyl)phenyl]-4(3)H-pyrimidinone;
5-(1-benzothien-2-yl)-2-(3-fluoro-2-hydroxyphenyl)-6-methyl-3-[4-(1-methylethyl)phenyl]-4(3H)-pyrimidinone;
2-(2-hydroxyphenyl)-6-methyl-3-[4-(1-methylethyl)phenyl]-5-(2-thienyl)-4(3H)-pyrimidinone;
2-(3-fluoro-2-hydroxyphenyl)-6-methyl-3-[4-(1-methylethyl)phenyl]-5-(2-thienyl)-4(3H)-pyrimidinone;
2-(2-hydroxyphenyl)-6-methyl-3-[4-(1-methylethyl)phenyl]-5-(5-methyl-2-thienyl)-4(3H)-pyrimidinone;
2-(3-fluoro-2-hydroxyphenyl)-6-methyl-3-[4-(1-methylethyl)phenyl]-5-(5-methyl-2-thienyl)-4(3H)-pyrimidinone;
5-(2,3-dihydro-1,4-benzodioxin-6-yl)-2-(2-hydroxyphenyl)-6-methyl-3-[4-(1-methylethyl)phenyl]-4(3H)-pyrimidinone; and
5-(2,3-dihydro-1,4-benzodioxin-6-yl)-2-(3-fluoro-2-hydroxyphenyl)-6-methyl-3-[4-(1-methylethyl)phenyl]-4(3H)-pyrimidinone.

Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. A preferred salt is a hydrochloride. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

The present invention provides compounds of Formula (I) above, which can be prepared using standard techniques. An overall strategy for preparing preferred compounds described herein can be carried out as described in this section. The examples, which follow, illustrate the synthesis of specific compounds. Using the protocols described herein as a model, one of ordinary skill in the art can readily produce other compounds of the present invention.

All reagents and solvents were obtained from commercial vendors. Starting materials were synthesized using standard techniques and procedures.

Synthesis Schemes

Compounds contained within this application have been prepared by the general methods detailed below in Scheme 1-3. Acylation of an enamine such as 2-amino-cyclohex-1-enecarboxylic acid ethyl ester (1) with and acyl chloride such as 2-benzyloxy-benzoyl chloride (2) provided the amide 3. Treatment of amide 3 with base effected cyclization to provide the tetrahydrobenzo[d][1,3]oxazin-4-one 4. Treatment of 4 with an amine such as 4-isopropylaniline (5) under acidic conditions provided the tetrahydroquinazolinone 6. Hydrogenolysis of the benzyl protecting group of 6 utilizing methods common to the art provided 7.

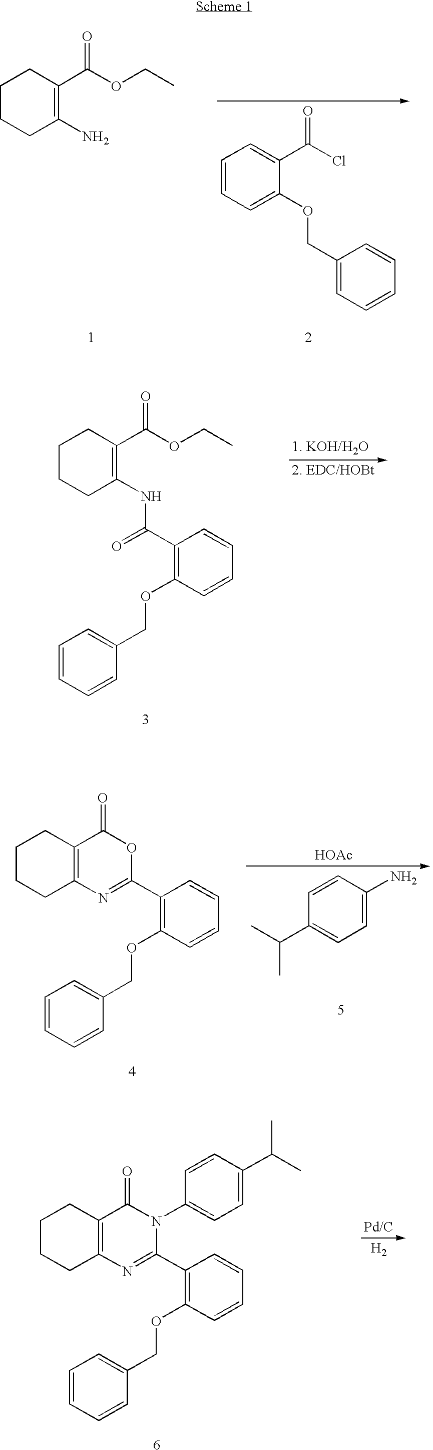

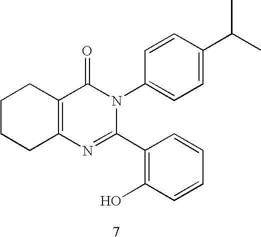

As outlined in Scheme 2, protection of the β-keto ester 8 followed by hydroysis of the ethyl ester utilizing standard basic conditions common to the art provided the acid 9. Conversion of the acid 9 to the acid chloride with oxalylchloride followed by treatment of this intermediate with an amine such as 4-isopropylaniline provided the β-keto amide 10. Treatment of 10 with ammonia and aluminun trichloride provided the intermediate enamine 11. Acylation of 11 with an acid chloride provided 12. Treatment of 12 under basic conditions provided pyrimidinone 13.

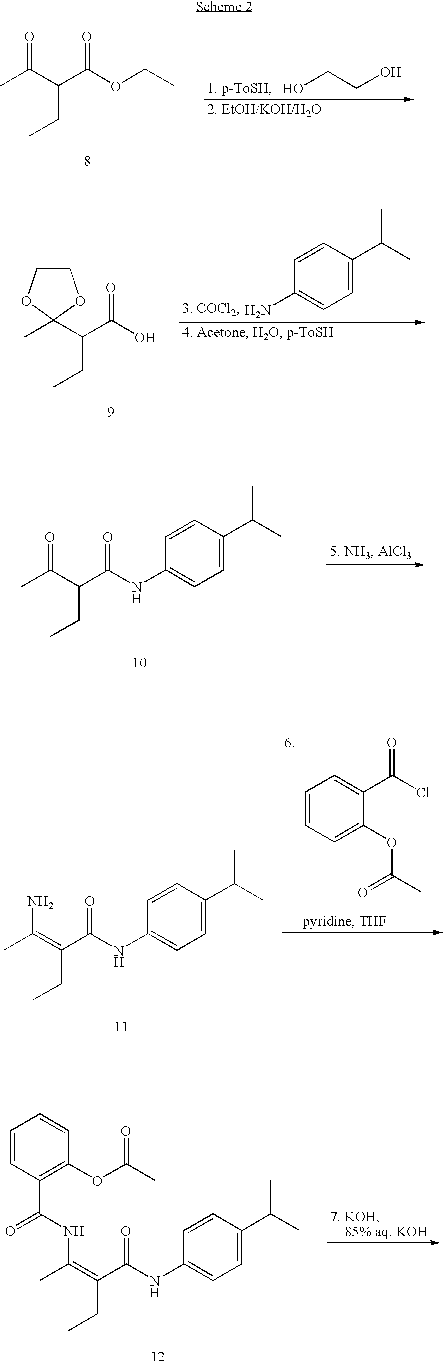

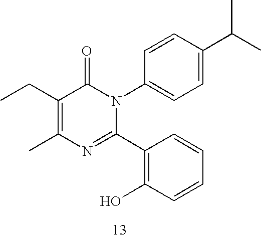

Alternatively, the C5 aryl pyrimidinones contained within this application may be prepared as outlined in Scheme 3. Acylation of the 3-amino crotonate 14 with an acid chloride provides the intermediate 16. Treatment of 16 with trimetylaluminum in the presence of an amine such as 17 provides the pyrimidinone 18. Bomination of 18 provides 19 which may be coupled with boronic acids such as phenyl boronic acid. Deprotection of the phenol with reagents that are common to the art provides the desired pyrimidinone 20.

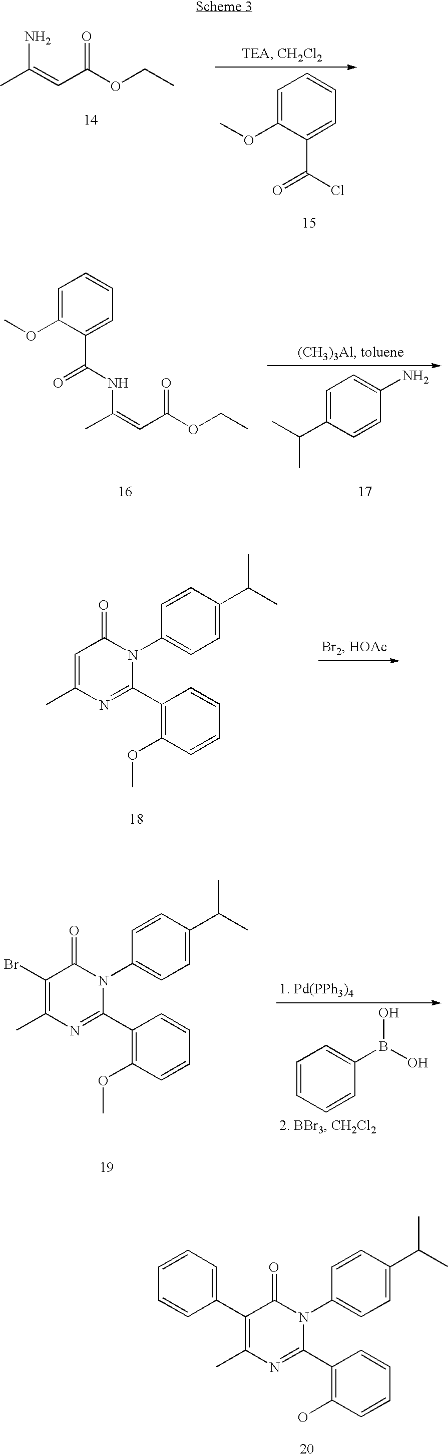

In order to use a compound of Formula (I) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably, the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered, for example, from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

In a preferred embodiment of the present invention, the present compounds are co-administered with an anti-resorptive agent. Such agents include, but are not limited estrogen, 1, 25 (OH)₂ vitamin D3, calcitonin, selective estrogen receptor modulators, vitronectin receptor antagonists, V-H+-ATPase inhibitors, src SH2 antagonists, bisphosphonates and cathepsin K inhibitors.

Another aspect of the present invention describes a method of treating a patient comprising administering to the patient an amount of a present compound sufficient to increase the serum PTH level. Preferably, the method is carried out by administering an amount of the compound effective to cause an increase in duration and/or quantity of serum PTH level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In an alternative embodiment of the present invention, the compound administered to a patient causes an increase in serum PTH having a duration of more than about twenty four hours provided that it is co-administered with an anti resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

Composition of Formula (I) and their pharmaceutically acceptable salts, which are active when given orally, can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) are demonstrated by the following tests:

(I) Calcium Receptor Inhibitor Assay

Calcilytic activity was measured by determining the IC₅₀ of the test compound for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 Suppl. 1:S483, 1995 (hereby incorporated by reference herein). Intracellular Ca²⁺ increases were elicited by increasing extracellular Ca²⁺ from 1 to 1.75 mM. Intracellular Ca²⁺ was measured using fluo-3, a fluorescent calcium indicator.

The procedure was as follows:

1. Cells were maintained in T-150 flasks in selection media (DMEM supplemented with 10% fetal bovine serum and 200 ug/mL hygromycin B), under 5% CO₂: 95% air at 37° C. and were grown up to 90% confluency.

2. The medium was decanted and the cell monolayer was washed twice with phosphate-buffered saline (PBS) kept at 37° C. After the second wash, 6 mL of 0.02% EDTA in PBS was added and incubated for 4 minutes at 37° C. Following the incubation, cells were dispersed by gentle agitation.

3. Cells from 2 or 3 flasks were pooled and pelleted (100×g). The cellular pellet was resuspended in 10-15 mL of SPF-PCB+ and pelleted again by centrifugation. This washing was done twice.

Sulfate- and phosphate-free parathyroid cell buffer (SPF-PCB) contains 20 mM Na-Hepes, pH 7.4, 126 mM NaCl, 5 mM KCl, and 1 mM MgCl₂. SPF-PCB was made up and stored at 4° C. On the day of use, SPF-PCB was supplemented with 1 mg/mL of D-glucose and 1 mM CaCl₂ and then split into two fractions. To one fraction, bovine serum albumin (BSA; fraction V, ICN) was added at 5 mg/mL (SPF-PCB+). This buffer was used for washing, loading and maintaining the cells. The BSA-free fraction was used for diluting the cells in the cuvette for measurements of fluorescence.

4. The pellet was resuspended in 10 mL of SPF-PCB+ containing 2.2 uM fluo-3 (Molecular Probes) and incubated at room temperature for 35 minutes.

5. Following the incubation period, the cells were pelleted by centrifugation. The resulting pellet was washed with SPF-PCB+. After this washing, cells were resuspended in SPF-PCB+ at a density of 1-2×106 cells/mL.

6. For recording fluorescent signals, 300 uL of cell suspension were diluted in 1.2 mL of SPF buffer containing 1 mM CaCl₂ and 1 mg/mL of D-glucose. Measurements of fluorescence were performed at 37° C. with constant stirring using a spectrofluorimeter. Excitation and emission wavelengths were measured at 485 and 535 nm, respectively. To calibrate fluorescence signals, digitonin (5 mg/mL in ethanol) was added to obtain Fmax, and the apparent Fmin was determined by adding Tris-EGTA (2.5 M Tris-Base, 0.3 M EGTA). The concentration of intracellular calcium was calculated using the following equation:
Intracellular calcium=(F−Fₘᵢₙ/Fₘₐₓ)×K_{d}; where K_{d}=400 nM.

7. To determine the potential calcilytic activity of test compounds, cells were incubated with test compound (or vehicle as a control) for 90 seconds before increasing the concentration of extracellular Ca²⁺ from 1 to 2 mM. Calcilytic compounds were detected by their ability to block, in a concentration-dependent manner, increases in the concentration of intracellular Ca²⁺ elicited by extracellular Ca²⁺.

In general, those compounds having lower IC₅₀ values in the Calcium Receptor Inhibitor Assay are more preferred compounds. Compounds having an IC₅₀ greater than 50 uM were considered to be inactive. Preferred compounds are those having an IC₅₀ of 10 uM or lower, more preferred compounds have an IC₅₀ of 1 uM, and most preferred compounds have an IC₅₀ of 0.1 uM or lower.

(II) Calcium Receptor Binding Assay

HEK 293 4.0-7 cells stably transfected with the Human Parathyroid Calcium Receptor (“HuPCaR”) were scaled up in T180 tissue culture flasks. Plasma membrane is obtained by polytron homogenization or glass douncing in buffer (50 mM Tris-HCl pH 7.4, 1 mM EDTA, 3 mM MgCl₂) in the presence of a protease inhibitor cocktail containing 1 uM Leupeptin, 0.04 uM Pepstatin, and 1 mM PMSF. Aliquoted membrane was snap frozen and stored at −80° C. ³H labeled compound was radiolabeled to a radiospecific activity of 44 Ci/mmole and was aliquoted and stored in liquid nitrogen for radiochemical stability.

A typical reaction mixture contains 2 nM ³H compound ((R,R)—N-4′-Methoxy-t-3-3′-methyl-1′-ethylphenyl-1-(1-naphthyl)ethylamine), or ³H compound (R)—N-[2-Hydroxy-3-(3-chloro-2-cyanophenoxy)propyl]-1,1-dimethyl-2-(4-methoxyphenyl)ethylamine 4-10 ug membrane in homogenization buffer containing 0.1% gelatin and 10% EtOH in a reaction volume of 0.5 mL. Incubation is performed in 12×75 polyethylene tubes in an ice water bath. To each tube 25 uL of test sample in 100% EtOH is added, followed by 400 uL of cold incubation buffer, and 25 uL of 40 nM ³H-compound in 100% EtOH for a final concentration of 2 nM. The binding reaction is initiated by the addition of 50 uL of 80-200 ug/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting.

Experimental Procedures

### EXAMPLE 1

### Preparation of 2-(2-hydroxy-phenyl)-3-(4-isopropyl-phenyl)-5,6,7,8-tetrahydro-3H-quinazolin-4-one

### a. 2-{[1-(2-Benzyloxy-phenyl)-methanoyl]-amino}-cyclohex-1-enecarboxylic acid ethyl ester

2-Amino-cyclohex-1-enecarboxylic acid ethyl ester (1.69 g, 10 mmol) and 2-benzyloxy-benzoyl chloride (2.47 g, 10 mmol) were dissolved in 300 ml CH₂Cl₂. To this mixture was added triethylamine (2.0 g, 20 mmol) and the reaction mixture was stirred overnight whereupon it was washed with H₂O; 1N HCl; H₂O and brine (100 ml each). The organic layer was concentrated and purified by flash column chromatography (20% ethyl acetate/hexane) to provide 3.7 g of the title compound.

### b. 2-(2-Benzyloxy-phenyl)-5,6,7,8-tetrahydrobenzo[d][1,3]oxazin-4-one

2-{[1-(2-Benzyloxy-phenyl)-methanoyl]-amino}-cyclohex-1-enecarboxylic acid ethyl ester (500 mg, 1.32 mmol) was dissolved in 30 ml EtOH. To this solution was added 85% KOH/H₂O (5 ml). This mixture was refluxed for 3 hours whereupon it was concentrated and the residue diluted in H₂O (20 ml). The pH of this mixture was adjusted to pH 2 with 1N HCl and extracted with CH₂Cl₂ (50 ml×3). The organic layers were combined and evaporated. The residue was dissolved in DMF and to this solution were added EDC (288 mg, 1.5 mmol), HOBT (202 mg, 1.5 mmol) and triethylamine (253 mg, 2.5 mmol). The reaction mixture was stirred overnight at room temperature whereupon the DMF was removed and the residue was dissolved in EtOAc, washed with 10% NaHCO₃, brine, dried over Na₂SO₄, filtered and concentrated. Column chromatography of the residue F.C.C. provided 270 mg of the title compound.

### c. 2-(2-Benzyloxy-phenyl)-3-(4-isopropyl-phenyl)-5,6,7,8-tetrahydro-3H-quinazolin-4-one

2-(2-Benzyloxy-phenyl)-5,6,7,8-tetrahydrobenzo[d][1,3]oxazin-4-one (150 mg, 0.45 mmol) was dissolved in 2 ml HOAc. To this solution was added 4-isopropyl-phenylamine (68 mg, 0.5 mmol) and heated at 100° C. for 1 hr. After cooling to R.T. the reaction mixture was poured into 20 ml H₂O and the pH of the mixture was adjusted to pH 4-5 using 6N NaOH. This mixture was then extracted with CH₂Cl₂ (50 ml×3). The organic layers were combined and washed with H₂O, brine, concentrated and purified by column chromatography (20% ethyl acetate/hexane) to provide 200 mg of the title compound.

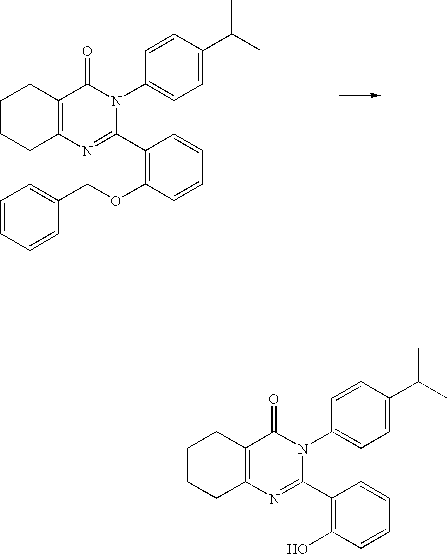

### d. 2-(2-Hydroxy-phenyl)-3-(4-isopropyl-phenyl)-5,6,7,8-tetrahydro-3H-quinazolin-4-one

2-(2-Benzyloxy-phenyl)-3-(4-isopropyl-phenyl)-5,6,7,8-tetrahydro-3H-quinazolin-4-one (200 mg, 0.44 mmol) was dissolved in 10 ml EtOH and degassed with argon. A catalytic amount of 10% Pd/C was added and H₂ balloon was applied. This mixture was stirred at R.T. for 5 hrs. whereupon it was filtered through Celite. The filtrate was concentrated and recrystallized from EtOAc/Hexane to provide 80 mg of the title compound. ¹H NMR (400 MHz, CDCl₃): □7.28-7.26(m, 2H), 7.17-7.10(m, 3H), 6.99-6.96(d, 1H), 6.65(d, 1H), 6.37(t, 1H), 2.95(m, 1H), 2.74-2.71(m, 2H), 2.62-2.59(m, 2H), 1.92-1.70(m, 4H), 1.57(s, 3H), 1.28(d, 6H). MS(m/z): 361.2(M+H).

### EXAMPLE 2

### 5-Ethyl-2-(2-hydroxy-phenyl)-3-(4-isopropyl-phenyl)-6-methyl-3H-pyrimidin-4-one

### a. 2-(2-Methyl-[1,3]dioxolan-2-yl)-butyric acid

A mixture of commercially available 2-ethyl-3-oxo-butyric acid ethyl ester (54 g, 0.34 mol), ethylene glycol (23.3 g, 0.375 mol), and p-toluenesulfonic acid (0.2 g) in toluene (500 mL) was heated to 120° C. for 4 h under a Dean-Stark apparatus. The reaction mixture was cooled to RT, the solvent was removed and the residue was partitioned between ethyl acetate and saturated NaHCO₃. The layers were separated, and the aqueous portion was extracted 3 times with ethyl acetate. The organic portions were pooled, dried (MgSO₄) and concentrated to provide (2-methyl-[1,3]dioxolan-2-yl)-acetic acid ethyl ester as a colorless oil in 91% yield (63 g).

To a solution of (2-methyl-[1,3]dioxolan-2-yl)-acetic acid ethyl ester (60 g, 0.297 mol) in EtOH (750 mL) was added 85% KOH solution in water (30 mL), and the mixture stirred at reflux overnight. The reaction mixture was cooled to room temperature, the solvent evaporated, and the residue was partitioned between CH₂Cl₂ and 2N HCl. After separating the layers, the aqueous portion was extracted 3 times with CH₂Cl₂. The organic portions were pooled, dried (Na₂SO₄), and concentrated in vacuo to provide the title compound as a light yellow oil (27 g, 52% yield).

### b. (4-Isopropyl-phenyl)-3-oxo-butyramide

To a 0° C. solution of 2-(2-Methyl-[1,3]dioxolan-2-yl)-butyric acid (6.5 g, 0.037 mol) in CH₂Cl₂ (50 mL) was added oxalyl chloride (11.7 mL) in a dropwise fashion. After 15 min at 0° C., the mixture was allowed to stir at RT for 2 h. The solvent and excess oxalyl chloride were removed to give an oil, which was brought up in fresh CH₂Cl₂ and cooled to 0° C. A pyridine solution (3 mL) of 4-isopropylaniline (3.0 g, 0.022 mol) was added dropwise, and the resulting solution was allowed to warm to RT while stirring overnight. The reaction mixture was partitioned between CH₂Cl₂ and 1N HCl. After separating the layers, the organic portion was washed with water and aq. NaHCO₃. The organic portion was pooled, dried (Na₂SO₄), and concentrated in vacuo to provide (4-isopropyl phenyl)-(2-2-methyl-[1,3]dioxolan-2-yl)-acetamide (3.5 g) which was used in the next reaction without further purification.

To a solution of (4-isopropyl phenyl)-2-(2-methyl-[1,3]dioxolan-2-yl)-acetamide (3.5 g, 0.012 mol) in acetone and water (50 mL/1 mL) was added p-toluenesulfonic acid (3.7 g, 0.019 mol). This mixture was stirred and heated to 95° C. for 4 h. After cooling to RT, the solvent was removed and the residue was partitioned between CH₂Cl₂ and aq. Na₂CO₃. After separating the layers, the aqueous layer was extracted 2 times with fresh CH₂Cl₂, and the combined organic portions were dried (Na₂SO₄), filtered and concentrated to provide the title compound as a white solid.

### c. (Z)-3-Amino-but-2-enoic acid (4-isopropyl-phenyl)-amide

A 0° C. solution of (4-isopropyl-phenyl)-3-oxo-butyramide (1.7 g, 6.9 mmol) in THF (250 mL) was saturated with gaseous ammonia for 3 h. AlCl₃ (1.4 g) was added and the mixture was allowed to warm to RT while stirring overnight. The resulting suspension was filtered, and the filtrate was concentrated to provide (Z)-3-amino-but-2-enoic acid (4-isopropyl-phenyl)-amide (1.6 g, 25%) which was used directly in the following reaction.

### d. Acetic acid 2-[(Z)-2-(4-isopropyl-phenylcarbamoyl)-1-methyl-vinyl carbamoyl-phenylester

To a solution of (Z)-3-amino-but-2-enoic acid (4-isopropyl-phenyl)-amide (0.8 g, 3.2 mmol) in THF (25 mL) and pyridine (1 mL) was added acetic acid 2-chlorocarbonyl-phenyl ester (0.77 g, 3.9 mmol). The mixture was heated to reflux for 3 hours. After cooling to RT, diethyl ether (200 mL) was added, and the precipitated salts were removed by filtration. The filtrate was concentrated, diluted with diethyl ether (250 mL), and washed three times with 1N HCl (100 mL portions). The organic layer was washed successively with water and brine, and dried over Na₂SO₄, filtered, and concentrated. Flash column chromatography of the residue provided the pure product 0.66 g of the title compound.

### e. 5-Ethyl-2-(2-hydroxy-phenyl)-3-(4-isopropyl-phenyl)-6-methyl-3H-pyrimidin-4-one

A solution of acetic acid 2-[(Z)-2-(4-isopropyl-phenylcarbamoyl)-1-methyl-vinylcarbamoyl-phenylester (0.4 g, 0.1 mmol) in EtOH (30 mL) and 85% KOH (5 mL) was heated to reflux for 5 hours. After cooling to RT, the reaction mixture was adjusted to pH 1 with 2N HCl and extracted three times with CH₂Cl₂. The organic portions were combined, dried (Na₂SO₄), filtered, and concentrated. Flash column chromatography of the residue (3% CH₃OH/CH₂Cl₂) provided the title compound as a white solid: ¹H NMR (400 MHz, CDCl₃): δ 7.28-7.26(m, 2H), 7.13-7.11(m, 3H), 7.10(d, 1H), 6.64(d, 1H), 6.38(t, 1H), 3.00-2.90(m, 1H), 2.64-^{2.62}(q, 2H), 2.44(s, 3H), 1.27-1.26(d, 6H), 1.21-1.17(t, 3H). MS(m/z): 349.2 (M+H).

### EXAMPLE 3

### Preparation of 5-ethyl-2-(3-fluoro-2-hydroxyphenyl)-6-methyl-3-[4-(1-methylethyl)phenyl]-4(3H)-pyrimidinone

Following the procedures for the preparation of 5-ethyl-2-(2-hydroxy-phenyl)-3-(4-isopropyl-phenyl)-6-methyl-3H-pyrimidin-4-one of Example 2 except substituting 2-(chlorocarbonyl)-6-fluorophenyl acetate for 2-(chlorocarbonyl)phenyl acetate of step 2D the title compound was prepared.

### EXAMPLE 4

### Preparation of 2-(2-hydroxyphenyl)-6-methyl-3-[4-(1-methylethyl)phenyl]-5-(2-methylpropyl)-4(3H)-pyrimidinone

### a. 4-methyl-2-(2-methyl-1,3-dioxolan-2-yl)-N-[4-(1-methylethyl)phenyl]pentanamide

4-methyl-2-(2-methyl-1,3-dioxolan-2-yl)pentanoic acid (3.26 g, 16.2 mmol) was dissolved in CH₂Cl₂ (15 mL), placed under N₂, and cooled to 0° C. Oxalyl chloride (2.0 M in CH₂Cl₂, 28 mL, 56.0 mmol) was added dropwise over 20 min. The reaction mixture was stirred at 0° C. for 30 min and then warmed to room temperature overnight. The reaction was then concentrated in vacuo. The resulting acid chloride was diluted with CH₂Cl₂ (15 mL) and cooled again to 0° C. A mixture of 4-isopropylaniline (4.1 mL, 30.0 mmol) and pyridine (2.1 mL, 26.0 mmol) was added dropwise over 6 min and the resulting reaction mixture stirred at 0° C. for 30 min before warming to room temperature for 3 days. The reaction was poured onto cold 1N HCl, diluted with CH₂Cl₂, and layers separated. The organic layer was washed successively with H₂O, sat. NaHCO₃, and brine. The organic layers were then dried over Na₂SO₄, filtered, and concentrated. The crude product was carried to the next step with no further purification: MS(ESI) 320.2 (M+H)⁺.

### b. 2-acetyl-4-methyl-N-[4-(1-methylethyl)phenyl]pentanamide

To a solution of 4-methyl-2-(2-methyl-1,3-dioxolan-2-yl)-N-[4-(1-methylethyl)phenyl]pentanamide (5.15 g, 16.1 mmol) in acetone (33 mL) and H₂O (1.0 mL) was added p-TsOH (4.91 g, 25.8 mmol). The reaction was heated at 95° C. for 17 h. The reaction mixture was then cooled to room temperature and concentrated in vacuo. The residue was diluted with CH₂Cl₂ and water and the aqueous layer was made basic (pH ˜10) with Na₂SO₃. The aqueous layer was then extracted three times with CH₂Cl₂ and the combined organic layers washed with H₂O and brine, dried over Na₂SO₄, filtered, and concentrated. Column chromatography (20% Ethyl acetate:hexane) afforded 1.31 g (30%) of 2-acetyl-4-methyl-N-[4-(1-methylethyl)phenyl]pentanamide: ¹H NMR (400 MHz, CHLOROFORM-d) δ ppm 7.97 (s, 1 H) 7.44 (d, J=8.59 Hz, 2 H) 7.20 (d, J=8.59 Hz, 2 H) 3.60-3.65 (m, 1 H) 2.89 (dt, J=13.83, 6.85 Hz, 1 H) 2.33 (s, 3 H) 1.83-1.88 (m, 1 H) 1.76-1.81 (m, 1 H) 1.62-1.70 (m, 1 H) 1.24 (d, J=6.82 Hz, 6 H) 0.98 (dd, J=6.44, 4.67 Hz, 6 H); MS(ESI) 276.4 (M+H)⁺.

### c. 2-(2-hydroxyphenyl)-6-methyl-3-[4-(1-methylethyl)phenyl]-5-(2-methylpropyl)-4(3H)-pyrimidinone

Salicylamide (0.363 g, 2.65 mmol) and Ti(Oi-Pr)₄ (3.4 mL, 11.6 mmol) were added to a solution of 2-acetyl-4-methyl-N-[4-(1-methylethyl)phenyl]pentanamide (0.603 g, 2.19 mmol) in xylene (12.5 mL) and the reaction was heated at reflux for 21 h. The reaction mixture was cooled to room temperature and concentrated in vacuo. The residue was diluted with CH₂Cl₂ and 1N HCl and stirred for 3 h. The aqueous layer was extracted 3 times with CH₂Cl₂. The combined organic layers were dried over Na₂SO₄, filtered, and concentrated. Column chromatography (5-25% Ethyl acetate:Hexane) yielded 0.176 g (21%) of the title compound as a white powder: ¹H NMR (400 MHz, CHLOROFORM-d) δ ppm 7.24-7.34 (m, 3 H) 7.08-7.18 (m, 3 H) 6.95-7.03 (m, 1 H) 6.67 (dd, J=8.21, 1.39 Hz, 1 H) 6.37-6.45 (m, 1 H) 2.89-2.99 (m, J=6.91, 6.91, 6.91, 6.91, 6.91, 6.91 Hz, 1 H) 2.43-2.51 (m, 5 H) 2.00-2.11 (m, J=13.63, 6.87, 6.87, 6.87, 6.69 Hz, 1 H) 1.23-1.30 (m, 6 H) 0.91-1.02 (m, 6 H); MS(ESI) 377.2 (M+H)⁺.

### EXAMPLE 5

### Preparation of 2-(3-fluoro-2-hydroxyphenyl)-6-methyl-3-[4-(1-methylethyl)phenyl]-5-(2-methylpropyl)-4(3H)-pyrimidinone

### a. 2-[3-fluoro-2-(methyloxy)phenyl]-6-methyl-3-[4-(1-methylethyl)phenyl]-5-(2-methylpropyl)-4(3H)-pyrimidinone

3-fluoro-2-hydroxybenzamide (0.175 g, 1.04 mmol) and Ti(O-i-Pr)₄ (1.6 mL, 5.29 mmol) were added to a solution of 2-acetyl-4-methyl-N-[4-(1-methylethyl)phenyl]pentanamide (0.281 g, 1.02 mmol) in xylene (5.0 mL) and the reaction was heated at reflux for 3 days. The reaction mixture was cooled to room temperature and concentrated in vacuo. The residue was diluted with CH₂Cl₂ and 1N HCl and stirred for 3 h. The aqueous layer was extracted 3 times with CH₂Cl₂. The combined organic layers were dried over Na₂SO₄, filtered, and concentrated. Column chromatography (2-20% Ethyl acetate:Hexane) yielded 0.077 g (18%) of 2-[3-fluoro-2-(methyloxy)phenyl]-6-methyl-3-[4-(1-methylethyl)phenyl]-5-(2-methylpropyl)-4(3H)-pyrimidinone as a white powder: MS(ESI) 409.2 (M+H)⁺.

### b. 2-[3-fluoro-2-(methyloxy)phenyl]-6-methyl-3-[4-(1-methylethyl)phenyl]-5-(2-methylpropyl)-4(3H)-pyrimidinone

To a cooled (0° C.) solution of 2-[3-fluoro-2-(methyloxy)phenyl]-6-methyl-3-[4-(1-methylethyl)phenyl]-5-(2-methylpropyl)-4(3H)-pyrimidinone (0.077 g, 0.189 mmol) in CH₂Cl₂ (2.0 mL) under N₂ was slowly added BBr₃ (1.0M in CH₂Cl₂, 0.49 mL, 0.49 mmol). The reaction was allowed to warm to room temperature overnight. Additional BBr₃ (0.17 mL, 0.17 mmol) was added and reaction mixture stirred for 4.5 h. The reaction was quenched with H₂O, diluted with CH₂Cl₂, and stirred. The aqueous layer was extracted 3 times with CH₂Cl₂. The combined organic layers were washed with brine, dried over Na₂SO₄, filtered, and concentrated. Column chromatography (2-25% Ethyl acetate:Hexane) produced 0.036 g (49%) of the title compound as a yellow solid: ¹H NMR (400 MHz, CHLOROFORM-d) δ ppm 7.26-7.32 (m, 3 H) 7.11 (d, J=8.34 Hz, 2 H) 6.96-7.06 (m, 1 H) 6.44 (d, J=8.34 Hz, 1 H) 6.35 (td, J=8.15, 4.93 Hz, 1 H) 2.90-3.00 (m, J=6.91, 6.91, 6.91, 6.91, 6.91, 6.91 Hz, 1 H) 2.42-2.54 (m, 5 H) 2.00-2.11 (m, J=13.47, 6.74, 6.74, 6.74, 6.74 Hz, 1 H) 1.23-1.31 (m, 6 H) 0.97-1.04 (m, 6 H); MS(ESI) 395.4 (M+H)⁺.

### EXAMPLE 6

### Preparation of 2-(2-hydroxyphenyl)-6-methyl-3-[4-(1-methylethyl)phenyl]-4(3H)-pyrimidinone

### a. N-[4-(1-methylethyl)phenyl]-3-oxobutanamide

A mixture of methyl acetoacetate (2.96 g, 25.5 mmol) and 4-isopropylaniline (1.16 mL, 8.48 mmol) was prepared and placed in a microwave reactor at 180° C. for 400 s. The resulting reaction mixture was purified via column chromatography (5-40% Ethyl acetate:Hexane) to yield 1.02 g (55%) of N-[4-(1-methylethyl)phenyl]-3-oxobutanamide: MS(ESI) 220.2 (M+H)⁺.

### b. 2-(2-hydroxyphenyl)-6-methyl-3-[4-(1-methylethyl)phenyl]-4(3H)-pyrimidinone

Salicylamide (0.904 g, 6.59 mmol) and Ti(Oi-Pr)₄ (6.7 mL, 22.9 mmol) were added to a solution of N-[4-(1-methylethyl)phenyl]-3-oxobutanamide (0.960 g, 4.38 mmol) in xylene (44 mL) and the reaction was heated at reflux for 21 h. The reaction mixture was cooled to room temperature and concentrated in vacuo. The residue was diluted with CH₂Cl₂ and 1N HCl and stirred for 22 h. The aqueous layer was extracted 3 times with CH₂Cl₂. The combined organic layers were dried over Na₂SO₄, filtered, and concentrated. Column chromatography (5-70% THF:hexane) yielded 0.185 g (13%) of the title compound as a white solid: ¹H NMR (400 MHz, CHLOROFORM-d) δ ppm 7.24-7.35 (m, 3 H) 7.08-7.19 (m, 3 H) 6.93-7.03 (m, 1 H) 6.63-6.72 (m, 1 H) 6.38-6.46 (m, 2 H) 2.94 (dt, J=13.83, 6.85 Hz, 1 H) 2.44 (s, 3 H) 1.18-1.30 (m, 6 H); MS(ESI) 377.2 (M+H)⁺.

### EXAMPLE 7

### Preparation of 2-(2-hydroxyphenyl)-5,6-dimethyl-3-[4-(1-methylethyl)phenyl]-4(3H-pyrimidinone

### a. 2-methyl-N-[4-(1-methylethyl)phenyl]-3-oxobutanamide

A mixture of ethyl 2-methylacetoacetate (2.68 g, 18.6 mmol) and 4-isopropylaniline (0.85 mL, 6.22 mmol) was prepared and placed in a microwave reactor at 180° C. for 600 s. The resulting reaction mixture was purified via column chromatography (5-40% Ethyl acetate:Hexane) to yield 0.740 g (51%) of 2-methyl-N-[4-(1-methylethyl)phenyl]-3-oxobutanamide: MS(ESI) 234.2 (M+H)⁺.

### b. 2-(2-hydroxyphenyl)-5,6-dimethyl-3-[4-(1-methylethyl)phenyl]-4(3H)-pyrimidinone

Salicylamide (0.710 g, 5.18 mmol) and Ti(Oi-Pr)₄ (5.4 mL, 18.4 mmol) were added to a solution of 2-methyl-N-[4-(1-methylethyl)phenyl]-3-oxobutanamide (0.790 g, 3.39 mmol) in xylene (34 mL) and the reaction was heated at reflux for 24 h. The reaction mixture was cooled to room temperature and concentrated in vacuo. The residue was diluted with CH₂Cl₂ and 1N HCl and stirred for 22 h. The aqueous layer was extracted 3 times with CH₂Cl₂. The combined organic layers were dried over Na₂SO₄, filtered, and concentrated. Column chromatography (5-70% THF:Hexane) yielded 0.238 g (21%) of the title compound as a white solid: ¹H NMR (400 MHz, CHLOROFORM-d) δ ppm 7.24-7.36 (m, 3 H) 7.08-7.17 (m, 3 H) 6.97 (d, J=8.08 Hz, 1 H) 6.66 (dd, J=8.21, 1.39 Hz, 1 H) 6.38-6.48 (m, 1 H) 2.89-2.99 (m, J=6.92, 6.92, 6.92, 6.92, 6.92 Hz, 1 H) 2.45 (s, 3 H) 2.15-2.22 (m, 3 H) 1.23-1.30 (m, 6 H); MS(ESI) 335.2 (M+H)⁺.

### EXAMPLE 8

### Preparation of 2-(2-hydroxyphenyl)-6-methyl-3-[4-(1-methylethyl)phenyl]-5-propyl-4(3H)-pyrimidinone

### a. 2-acetyl-N-[4-(1-methylethyl)phenyl]-4-pentenamide

A mixture of ethyl 2-acetyl-4-pentenoate (2.25 g, 14.4 mmol) and 4-isopropylaniline (0.66 mL, 4.83 mmol) was prepared and placed in a microwave reactor at 180° C. for 600 s. The resulting reaction mixture was purified via column chromatography (5-40% Ethyl acetate:Hexane) to yield 0.620 g (50%) of 2-acetyl-N-[4-(1-methylethyl)phenyl]-4-pentenamide: MS(ESI) 260.2 (M+H)⁺.

### a. 2-acetyl-N-[4-(1-methylethyl)phenyl]pentanamide

A solution of 2-acetyl-N-[4-(1-methylethyl)phenyl]-4-pentenamide (0.570 g, 2.20 mmol) in ethanol (11 mL) and ethyl acetate (11 mL) was purged with N₂ for 5 min. Pd/C (10%, 0.123 g) was added, reaction flask evacuated, and reaction mixture stirred under H₂ (balloon pressure) for 19 h. The reaction was filtered through a Celite-plugged filter frit, washed with CH₃OH and CH₂Cl₂, and concentrated to yield 0.580 g of crude compound which was carried to the next step: MS(ESI) 262.6 (M+H)⁺.

### c. 2-(2-hydroxyphenyl)-6-methyl-3-[4-(1-methylethyl)phenyl]-5-propyl-4(3H)-pyrimidinone

Salicylamide (0.381 g, 2.78 mmol) and Ti(Oi-Pr)₄ (3.2 mL, 10.9 mmol) were added to a solution of 2-acetyl-N-[4-(1-methylethyl)phenyl]pentanamide (0.574 g, 2.20 mmol) in xylene (22 mL) and the reaction was heated at reflux for 19 h. The reaction mixture was cooled to room temperature and concentrated in vacuo. The residue was diluted with CH₂Cl₂ and 1N HCl and stirred for 2 days. The aqueous layer was extracted 3 times with CH₂Cl₂. The combined organic layers were dried over Na₂SO₄, filtered, and concentrated. Column chromatography (R,R-Whelko, 95:5 EtOH:Hexane) yielded 0.177 g (22%) of the title compound as a white solid: ¹H NMR (400 MHz, CHLOROFORM-d) δ ppm 7.24-7.34 (m, 3 H) 7.09-7.19 (m, 3 H) 6.97 (d, J=8.34 Hz, 1 H) 6.67 (d, J=8.08 Hz, 1 H) 6.41 (t, J=7.71 Hz, 1 H) 2.93 (dt, J=13.71, 6.92 Hz, 1 H) 2.51-2.63 (m, 2 H) 2.44-2.51 (m, 3 H) 1.62 (ddd, J=15.16, 7.45, 7.20 Hz, 2 H) 1.25 (d, J=6.82 Hz, 6 H) 1.00-1.11 (m, 3 H); MS(ESI) 363.2 (M+H)⁺.

### EXAMPLE 9

### Preparation of 5-butyl-2-(2-hydroxyphenyl)-6-methyl-3-[4-(1-methylethyl)phenyl]-4(3H)-pyrimidinone

### a. 2-acetyl-N-[4-(1-methylethyl)phenyl]hexanamide

A mixture of ethyl 2-n-butylacetoacetate (2.79 g, 15.0 mmol) and 4-isopropylaniline (0.68 mL, 4.97 mmol) was prepared and placed in a microwave reactor at 180° C. for 600 s. The resulting reaction mixture was purified via column chromatography (5-40% Ethyl acetate:Hexane) to yield 0.840 g (61%) of 2-acetyl-N-[4-(1-methylethyl)phenyl]hexanamide: MS(ESI) 276.2 (M+H)⁺.

### b. 5-butyl-2-(2-hydroxyphenyl)-6-methyl-3-[4-(1-methylethyl)phenyl]-4(3H)-pyrimidinone

Salicylamide (0.512 g, 3.73 mmol) and Ti(Oi-Pr)₄ (4.6 mL, 15.7 mmol) were added to a solution of 2-acetyl-N-[4-(1-methylethyl)phenyl]hexanamide (0.840 g, 3.05 mmol) in xylene (30 mL) and the reaction was heated at reflux for 19 h. The reaction mixture was cooled to room temperature and concentrated in vacuo. The residue was diluted with CH₂Cl₂ and 1N HCl and stirred for 2 days. The aqueous layer was extracted 3 times with CH₂Cl₂. The combined organic layers were dried over Na₂SO₄, filtered, and concentrated. Column chromatography (R,R-Whelko, 95:5 EtOH:Hexane) yielded 0.276 g (24%) of the title compound as a white solid: ¹H NMR (400 MHz, CHLOROFORM-d) δ ppm 7.22-7.30 (m, 3 H) 7.09-7.18 (m, 3 H) 6.90-7.00 (m, 1 H) 6.63-6.70 (m, 1 H) 6.41 (t, J=7.71 Hz, 1 H) 2.88-2.99 (m, J=6.86, 6.86, 6.86, 6.86, 6.86, 6.86 Hz, 1 H) 2.54-2.63 (m, 2 H) 2.42-2.49 (m, 3 H) 1.51-1.60 (m, 2 H) 1.44 (dq, J=14.53, 7.28 Hz, 2 H) 1.22-1.30 (m, 6 H) 0.88-1.00 (m, 3 H); MS(ESI) 377.2 (M+H)⁺.

### EXAMPLE 10

### Preparation of 2-(2-hydroxyphenyl)-6-methyl-3-[4-(1-methylethyl)phenyl]-5-phenyl-4(3H)-pyrimidinone

### a. Ethyl (2Z)-3-({[2-(methyloxy)phenyl]carbonyl}amino)-2-butenoate

To a solution of ethyl 3-aminocrotonate (20.0 mL, 0.158 mol) in CH₂Cl₂ (500 mL) were added anisoyl chloride (21.5 mL, 0.160 mol) and triethylamine (44 mL, 0.316 mol) and reaction mixture stirred for 21 h. The reaction was washed with H₂O, 1N HCl, H₂O, and brine. The organic layer was dried over Na₂SO₄, filtered, and concentrated. Column chromatography (1-20% Ethyl acetate:Hexane) yielded 15.2 g (36%) of ethyl (2Z)-3-({[2-(methyloxy)phenyl]carbonyl}amino)-2-butenoate: 1H NMR (400 MHz, CHLOROFORM-d) δ ppm 8.10 (dd, J=7.83, 1.77 Hz, 1 H) 7.47-7.54 (m, 1 H) 7.04-7.14 (m, 1 H) 7.02 (d, J=8.59 Hz, 1 H) 5.01 (d, J=1.01 Hz, 1 H) 4.15-4.25 (m, 2 H) 4.00-4.10 (m, 3 H) 2.48-2.56 (m, 3 H) 1.27-1.33 (m, 3 H); MS(ESI) 264.2 (M+H)⁺.

### b. 6-methyl-3-[4-(1-methylethyl)phenyl]-2-[2-(methyloxy)phenyl]-4(3H)-pyrimidinone

Trimethylaluminum (2.0M in Hexane, 2.25 mL, 4.50 mmol) was added to a solution of 4-isopropylaniline (0.63 mL, 4.61 mmol) in toluene (38 mL) under N₂. The reaction was stirred for 35 min. ethyl (2Z)-3-({[2-(methyloxy)phenyl]carbonyl}amino)-2-butenoate (1.00 g, 3.83 mmol) was added and reaction heated at reflux for 16 h. The reaction mixture was cooled to room temperature and concentrated in vacuo. The residue was diluted with CH₂Cl₂ and washed with H₂O and brine. The organic layer was dried over Na₂SO₄, filtered, and concentrated. Purification by column chromatography (2-30% Ethyl acetate:Hexane) afforded 0.280 g (22%) of 6-methyl-3-[4-(1-methylethyl)phenyl]-2-[2-(methyloxy)phenyl]-4(3H)-pyrimidinone: MS(ESI) 335.2 (M+H)⁺.

### c. 5-bromo-6-methyl-3-[4-(1-methylethyl)phenyl]-2-[2-(methyloxy)phenyl]-4(3H)-pyrimidinone

Dropwise addition of bromine (0.084 mL, 1.63 mmol) to a solution of 6-methyl-3-[4-(1-methylethyl)phenyl]-2-[2-(methyloxy)phenyl]-4(3H)-pyrimidinone (0.280 g, 0.838 mmol) in glacial acetic acid (8.0 mL) was done over 4 min. The reaction mixture was stirred overnight. The reaction was then diluted with CH₂Cl₂ and washed with H₂O, sat. NaHCO₃, and brine. The organic layer was dried over Na₂SO₄, filtered, and concentrated. Column chromatography (0-5% CH₃OH:CH₂Cl₂) produced 0.125 g (36%) of 5-bromo-6-methyl-3-[4-(1-methylethyl)phenyl]-2-[2-(methyloxy)phenyl]-4(3H)-pyrimidinone: ¹H NMR (400 MHz, CHLOROFORM-d) δ ppm 7.21-7.30 (m, 2 H) 7.16 (s, 1 H) 6.91 (t, J=7.45 Hz, 2 H) 6.73 (s, 1 H) 6.59 (d, J=8.34 Hz, 1 H) 3.53-3.61 (m, 3 H) 2.81 (dt, J=13.83, 6.85 Hz, 1 H) 2.59-2.65 (m, 3 H) 1.65 (s, 1 H) 1.13-1.19 (m, 6 H); MS(ESI) 415.0 (M+H)⁺.

### d. 6-methyl-3-[4-(1-methylethyl)phenyl]-2-[2-(methyloxy)phenyl]-5-phenyl-4(3H)-pyrimidinone

To a solution of 5-bromo-6-methyl-3-[4-(1-methylethyl)phenyl]-2-[2-(methyloxy)phenyl]-4(3H)-pyrimidinone (0.120 g, 0.290 mmol) in degassed 1,4-dioxane (3.0 mL) were added phenylboronic acid (0.074 g, 0.607 mmol) in EtOH (0.5 mL), sodium carbonate (0.062 g, 0.585 mmol) in H₂O (0.5 mL), and Pd(PPh₃)₄ (0.036 g, 0.031 mmol). The heterogeneous reaction mixture was stirred vigorously for 10 min and then placed in a microwave reactor at 180° C. for 700 s. The reaction mixture was then filtered through a Celite-plugged filter frit, washed with CH₃OH and CH₂Cl₂, and concentrated. The residue was diluted with ethyl acetate, washed with H₂O, dried over Na₂SO₄, filtered, and concentrated. The resulting crude product was carried to the next step: MS(ESI) 411.2 (M+H)⁺.

### e. 2-(2-hydroxyphenyl)-6-methyl-3-[4-(1-methylethyl)phenyl]-5-phenyl-4(3H)-pyrimidinone

To a cooled (0° C.) solution of 6-methyl-3-[4-(1-methylethyl)phenyl]-2-[2-(methyloxy)phenyl]-5-phenyl-4(3H)-pyrimidinone (0.119 g, 0.290 mmol) in CH₂Cl₂ (3.2 mL) under N₂ was slowly added BBr₃ (1.0M in CH₂Cl₂, 0.60 mL, 0.600 mmol). The reaction was allowed to warn to room temperature overnight. Additional BBr₃ (1.5 mL, 1.50 mmol) was added and reaction mixture stirred for 5 h. The reaction was quenched with sat. NaHCO₃ and extracted with CH₂Cl₂. The organic layer was washed with brine, dried over NA₂SO₄, filtered, and concentrated. Column chromatography (2-50% Ethyl acetate:Hexane) produced 0.059 g (51%) of the title compound as a white solid: ¹H NMR (400 MHz, CHLOROFORM-D) δ ppm 7.36-7.48 (m, 5 H) 7.24-7.34 (m, 3 H) 7.14-7.21 (m, 3 H) 7.00-7.08 (m, 1H) 6.71-6.79 (m, 1 H) 6.45 (t, J=7.71 Hz, 1 H) 2.93 (ddd, J=13.77, 7.07, 6.95 Hz, 1 H) 2.37-2.45 (m, 3 H) 1.22-1.30 (m, 6 H); MS(ESI) 397.2 (M+H)⁺.

### EXAMPLE 11

### Preparation of 5-(1-benzothien-2-yl)-2-(2-hydroxyphenyl)-6-methyl-3-[4-(1-methylethyl)phenyl]-4(3H)-pyrimidinone

Following the procedures for the preparation of 2-(2-hydroxyphenyl)-6-methyl-3-[4-(1-methylethyl)phenyl]-5-phenyl-4(3H)-pyrimidinone of Example 10 except substituting 2-benzothiophene boronic acid for phenylboronic acid of step 10 d the title compound was prepared: MS(ESI) 453.2 (M+H)⁺

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound according to formula (I) hereinbelow:
The compounds of the present invention are selected from Formula (I) herein below: 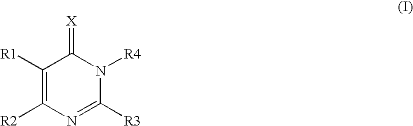
wherein:
R1 and R2 may each be independently selected from the group consisting of H, halogen, CN, alkyl, alkyl-aryl, aryl, substituted aryl, hetero aryl and substituted heteroaryl
or R1 and R2 may be bonded together to form a carbocylic ring, heterocylic ring, aryl or heteroaryl ring
R3 is an aryl group or heteroaryl group which may have 1-5 substituents each selected from the group consisting of H, halogen, CN, CF₃, OCF₃, alkyl, alkoxy, OC(O)alkyl or OH
R4 is an aryl group which may have 1 to 3 substituents consisting of H, halogen, CN, CF₃, alkyl, substituted alkyl and alkoxy; and
X is oxygen or sulphur.

2. A compound according to claim 1 selected from the group consisting of:
2-(2-hydroxyphenyl)-3-[4-(1-methylethyl)phenyl]-5,6,7,8-tetrahydro-4(3H)-quinazolinone
5-ethyl-2-(2-hydroxyphenyl)-6-methyl-3-[4-(1-methylethyl)phenyl]-4(3H)-pyrimidinone
5-ethyl-2-(3-fluoro-2-hydroxyphenyl)-6-methyl-3-[4-(1-methylethyl)phenyl]-4(3H)-pyrimidinone
2-(2-hydroxyphenyl)-6-methyl-3-[4-(1-methylethyl)phenyl]-5-(2-methylpropyl)-4(3H)-pyrimidinone
2-(3-fluoro-2-hydroxyphenyl)-6-methyl-3-[4-(1-methylethyl)phenyl]-5-(2-methylpropyl)-4(3H)-pyrimidinone
2-(2-hydroxyphenyl)-6-methyl-3-[4-(1-methylethyl)phenyl]-4(3H)-pyrimidinone
2-(2-hydroxyphenyl)-5,6-dimethyl-3-[4-(1-methylethyl)phenyl]-4(3H)-pyrimidinone
2-(2-hydroxyphenyl)-6-methyl-3-[4-(1-methylethyl)phenyl]-5-propyl-4(3H)-pyrimidinone
5-butyl-2-(2-hydroxyphenyl)-6-methyl-3-[4-(1-methylethyl)phenyl]-4(3H)-pyrimidinone
2-(2-hydroxyphenyl)-6-methyl-3-[4-(1-methylethyl)phenyl]-5-phenyl-4(3H)-pyrimidinone
5-(1-benzothien-2-yl)-2-(2-hydroxyphenyl)-6-methyl-3-[4-(1-methylethyl)phenyl]-4(3H)-pyrimidinone
5-(1-benzothien-2-yl)-2-(3-fluoro-2-hydroxyphenyl)-6-methyl-3-[4-(1-methylethyl)phenyl]-4(3H)-pyrimidinone
2-(2-hydroxyphenyl)-6-methyl-3-[4-(1-methylethyl)phenyl]-5-(2-thienyl)-4(3H)-pyrimidinone
2-(3-fluoro-2-hydroxyphenyl)-6-methyl-3-[4-(1-methylethyl)phenyl]-5-(2-thienyl)-4(3H)-pyrimidinone
2-(2-hydroxyphenyl)-6-methyl-3-[4-(1-methylethyl)phenyl]-5-(5-methyl-2-thienyl)-4(3H)-pyrimidinone
2-(3-fluoro-2-hydroxyphenyl)-6-methyl-3-[4-(1-methylethyl)phenyl]-5-(5-methyl-2-thienyl)-4(3H)-pyrimidinone
5-(2,3-dihydro-1,4-benzodioxin-6-yl)-2-(2-hydroxyphenyl)-6-methyl-3-[4-(1-methylethyl)phenyl]-4(3H)-pyrimidinone; and
5-(2,3-dihydro-1,4-benzodioxin-6-yl)-2-(3-fluoro-2-hydroxyphenyl)-6-methyl-3-[4-(1-methylethyl)phenyl]-4(3H)-pyrimidinone.

3. A method of antagonizing a calcium receptor, which comprises administering to a subject in need thereof, an effective amount of a compound according to claim 1.

4. A method of treating a disease or disorder characterized by an abnormal bone or mineral homeostasis, which comprises administering to a subject in need of treatment thereof an effective amount of a compound of claim 1.

5. A method according to claim 4 wherein the bone or mineral disease or disorder is selected from the group consisting of osteosarcoma, periodontal disease, fracture healing, osteoarthritis, joint replacement, rheumatoid arthritis, Paget's disease, humoral hypercalcemia, malignancy and osteoporosis.

6. A method according to claim 5 wherein the bone or mineral disease or disorder is osteoporosis.

7. A method according to claim 6 wherein the compound is co-administered with an anti-resorptive agent.

8. A method according to claim 7 wherein the anti-resorptive agent is selected from the group consisting of estrogen, 1, 25 (OH)₂ vitamin D3, calcitonin, selective estrogen receptor modulators, vitronectin receptor antagonists, V-H+-ATPase inhibitors, src SH2 antagonists, bisphosphonates and cathepsin K inhibitors.

9. A method of increasing serum parathyroid levels which comprises administering to a subject in need of treatment an effective amount of a compound of claim 1.

10. A method according to claim 9 wherein the compound is co-administered with an anti-resorptive agent.

11. A method according to claim 10 wherein the anti-resorptive agent is selected from the group consisting of: estrogen, 1, 25 (OH)₂ vitamin D3, calcitonin, selective estrogen receptor modulators, vitronectin receptor antagonists, V-H+-ATPase inhibitors, src SH2 antagonists, bisphosphonates and cathepsin K inhibitors.
